(19) United States
(12) Patent Application Publication
(10) Pub. No.: US 2024/0001035 A1
(43) Pub. Date: Jan. 4, 2024

(54) FILTER NEEDLE

(71) Applicant: Terumo Europe N.V., Leuven (BE)

(72) Inventors: Ludo DANIELS, Tongeren (BE); Pieter CASTELEYN, Herent (BE); Christian FRIPON, Haasrode (BE)

(21) Appl. No.: 18/038,737

(22) PCT Filed: Nov. 24, 2021
(86) PCT No.: PCT/EP2021/082896

§ 371 Date: May 25, 2023

(30) Foreign Application Priority Data
Nov. 25, 2020 (BE) 2020/5851

(51) Int. Cl. A61M 5/31

(52) U.S. Cl. CPC A61M 5/3145

(57) ABSTRACT

Filter needle comprising a hub having a proximal end arranged for removable connection to a syringe body, and having a distal end in which a needle is mounted, the hub provided with a channel between the proximal end and the distal end, for when the syringe body is connected, establishing a fluid connection between the syringe body and the needle, wherein a filter unit is mounted in the channel of the hub.

### FIELD OF THE INVENTION

The invention generally relates to medical equipment and, more specifically, to a filter needle for use in medical injection equipment such as hypodermic needles.

A medical injection equipment, such as a syringe is generally used for directly injecting a medical liquid into a patient. The syringe includes a syringe body and a syringe needle separably installed or mounted to a front distal end of the syringe body.

### BACKGROUND TO THE INVENTION

In recent years increasing concern has been expressed about the presence of particulate contamination in medical solutions infused or injected into patients, and about the possible harm such contamination may cause to the patient.

There are a variety of possibilities that particulate contamination, such as solid impurities, may be admitted into the syringes or into the medical solutions stored therein. These impurities may include not only the dust or fine particles which have ingressed into the syringes from external sources but also the fine fragments of glass or rubber which are produced when an ampoule or other breakable container of a medical solution is cut open or during the process in which a rubber plug is fitted to the liquid reservoir. The solid impurities thus present in the medical solution in a syringe will find their way through the needle holder device and the injection needle into the bloodstreams or body tissues together with the solution injected thereinto and may injure the vascular or other tissues.

Particularly sensitive are injections of medical liquid directly into the human eye. Medical liquids or drugs for such ophthalmic applications, such as biopharmaceuticals, are usually very sensitive and contaminants can easily form. Such contamination and/or impurities should not enter the patient; there is a risk that it will never get out of the eyeball and cause sight problems.

It is thus apparent that there is a need in the medical field for some means to prevent or minimize particulate matter contamination in medical solutions, in the drawing-up of medical solutions from the vial as well as during injection of the medical solution into the patient. It has therefore been suggested that filters be employed in injection equipment to filter particulate contamination from medical solutions fed to patients.

Various types of filter needles have been proposed in recent years, including a filter or filter assembly installed in a fixing member (hub) for fixing a needle in order to separably install a syringe needle to the front of the syringe.

KR 101 833 729 B1 discloses a filter for a medical fluid injection device, comprised of two threated portions screwed together to form an injection hole formed with a filtration space, containing an injection liquid filtering material, and placed on a syringe to filter out lumps contained in the injection liquid during injection of medical liquids.

The nature of the filter material described in the prior art can be varying such as paper, foam, rubber, various synthetic resin, porous ceramics or woven fabric, membrane.

In some prior art methods the filter device is engaged with the hub by a fusing/melting method (e.g. ultrasone fusing) or a method of using a bonding, a hot melt, etc. In such cases the material used as an adhesive should be harmless to a human body in terms of a chemical reaction with a medical fluid.

Such filters or filter devices need to satisfy various requirements. A filter or filter device should, of course, filter a fluid flowing therethrough to prevent particles of a predefined size to pass through the filter. The filter or filter device may not have leaks. That is, no fluid may bypass the filter, since the bypassed or leaked fluid is not filtered. The filter or filter device preferably has as little influence as possible on the fluid flow. More specifically, the filter or filter device has to have a minimal influence on flow rate and preferably causes a minimal pressure loss. A low flow rate or high pressure loss might cause a user of the filter device to apply more pressure in order to speed up the fluid flow. This might result in fluid leaving the needle at a too high speed or under a too high pressure, which might cause damage to tissue wherein the fluid is injected. This can be especially dangerous when the fluid is an ophthalmic solution being injected into a human eye. Also, when the filter influences the fluid flow or pressure loss too much, this may prevent the user from using a filter needle at all, with consequently health risks. These requirements can be mutually conflicting, and a trade-off is often required to balance these mutually conflicting requirements whilst remaining cost effective. The cost of the equipment is in itself an important requirement, particularly in view of the fact that most of the injection equipment in use today is disposable, i.e. designed to be discarded after a single use.

There is therefore a need for a filter needle device obviating at least one of the above mentioned problems. Moreover, there is a need for a filter needle device satisfying as much as possible of the efficiency, cost and compatibility requirements discussed above.

### SUMMARY OF THE INVENTION

According to an aspect of the invention, there is provided a filter needle comprising a hub having a proximal end arranged for removable connection to a syringe body, and having a distal end in which a needle is mounted, the hub provided with a channel between the proximal end and the distal end, for when the syringe body is connected, establishing a fluid connection between the syringe body and the needle, wherein a filter unit is mounted in the channel of the hub, wherein the filter unit comprising a sheet of woven filter material, wherein the sheet of woven filter material is fixated to surrounding material, wherein surrounding material protrudes through the sheet of woven filter material.

By providing a sheet of filter material through which the surrounding material protrudes, a firm and reliable connection can be obtained. Thus leakage and/or rupture can be minimized or obviated.

In an embodiment, the surrounding material is hub material. This allows the sheet of filter material to be directly fixated to the hub, which provides for an improved connection of the sheet of filter material than a mechanical connection.

Advantageously, the surrounding material protrudes through meshes of the filter material. By allowing the surrounding material to flow, in liquid form, through meshes of the filter material, such that, after solidification a protrusion of the surrounding material through the filter material is obtained, a firm and reliable connection can be achieved between the filter material and the surrounding material. For example, the surrounding material can be locally melted to allow it to flow through the filter material. Alternatively, moulding can be envisaged, in which the sheet of filter material is placed in a mould and the surrounding material is moulded around, and partially through, it.

Alternatively, the filter unit comprises a sleeve, in particular a tubular sleeve, and the surrounding material is then the material of the sleeve. The sleeve material then protrudes through the sheet of filter material providing for a firm and reliable connection.

Advantageously, the surrounding material is engaged with the sheet of filter material over the entire circumference of the sheet of filter material. Preferably, the surrounding material is engaged with an outer edge of the sheet of filter material.

The filter unit can thus be a sheet of filter material, that is directly connected to the hub, in particular to the hub material.

The filter unit can also be a sheet of filter material and a sleeve, in which the sheet of filter material is fixated to the sleeve, in particular to the sleeve material. Then, the filter unit is positioned in the channel of the hub.

The sleeve is advantageously a tubular sleeve. Then, the sheet of filter material preferably is disc shaped.

Advantageously, the sheet of filter material has a diameter that is larger than an inner diameter of the sleeve or than an inner diameter of the channel of the hub, to provide for sufficient contact surface of the outer edge of the filter material with the surrounding material of the sleeve or hub respectively. Thus, a sufficient contact surface and connecting surface is provided to achieve a sufficiently firm connection. Preferably, the diameter of the sheet of filter material is not larger than an outer diameter of the sleeve or an outer diameter of the hub. Thus, it may be prevented that the sheet of filter material extends outside of the sleeve or hub respectively. This may require an additional step to cut off the sheet, and/or may introduce a vulnerability to the connection between the sheet of filter material and the surrounding material.

Using a woven filter has advantages in relation to using, for example, a membrane filter. A woven material is, for example, less sensitive to tearing and may create a lower pressure loss than a membrane filter. Thus, providing a woven filter material, may have less impact on the flow, as well as may cause less pressure loss, while the woven filter material may have meshes preventing the particles of a predefined size to pass through them.

Instead of using a tubular sleeve to which the sheet of filter material is integrally bonded by allowing material of the sleeve to protrude through the filter material, the sheet of filter material can directly be fixated to the hub. Then, material of the hub can locally melted to allow it to flow through the sheet of filter material such that, after solidification, the material of the hub protrudes through the sheet of filter material, in particular through meshes of the sheet of filter material. Then, also a firm and reliable connection between the sheet of filter material and the hub directly can be obtained. Such connection may prevent and/or obviate leakage and/or rupture at the connection of the filter material to the hub.

Furthermore, a filter unit comprising a tubular sleeve and a sheet of woven filter material fixated in the tubular sleeve can be seen as relatively simple. The simplicity of the filter unit may allow for an efficient and cost-effective mass production of the filter unit. Furthermore, by providing a filter unit with a tubular sleeve, a filter unit with a common form is achieved, that may be relatively easy to handle and/or to manipulate. As such, the filter unit can easily be used in combination with, for example, existing hubs without requiring, any or too many, additional changes to be made to these existing hubs.

Alternatively or additionally, the sheet of woven filter material may be positioned halfway of the height of the tubular sleeve. By providing the sheet of woven filter material halfway the height of the tubular sleeve, a symmetrical filter unit may be achieved. As such, it doesn't matter which end of the filter unit is inserted first into the hub. Someone attempting to insert the filter unit into the hub therefore doesn't have to check whether the filter unit is positioned correctly, resulting in an easy and effortless insertion of the filter unit into the hub. As such, inserting the filter unit into the channel of the hub, can be done in a robust and reliable manner. The tubular sleeve of the filter unit is to be inserted in line with the channel of the hub, but the orientation of the filter unit, which end of the filter unit is positioned first into the channel, is not relevant for the insertion of the filter unit in the channel, thereby a robust and failure-proof assembly can be obtained.

Alternatively or additionally, the sheet of woven filter material may be disc-shaped having an outer diameter that is larger than an inner diameter of the tubular sleeve, such that an outer edge of the sheet of woven filter material is enclosed by the tubular sleeve. Thus a firm connection between the sheet of woven filter material and the tubular sleeve can be obtained. Advantageously, the entire outer edge of the sheet of woven material is enclosed by the tubular sleeve. Preferably, the outer edge of the sheet of woven material is over its entire circumference, preferably over an annular ring of the outer edge, enclosed by the tubular sleeve. By enclosing the entire circumference, leakage between the tubular sleeve and the filter can be avoided. Preferably, the outer edge of the sheet of woven material is such enclosed by the tubular sleeve that the material of the tubular sleeve protrudes through meshes of the woven material. As such a reliable and firm connection can be obtained between the sheet of woven material and the sleeve, thereby preventing leakage and/or rupture.

Alternatively or additionally, the filter unit, in particular the tubular sleeve, may be elastic. An elastic filter unit can be deformed elastically when pressure is applied to the filter unit. This elastic deformation can close possible holes through which a medical solution could bypass the filter. As such, leakage between the filter unit and the channel wall can be prevented, as the elastically deformable sleeve may tightly fit into the channel wall and/or may fill any irregularities of the sleeve wall and/or the channel wall thus preventing leakage. Furthermore, insertion of the filter unit into the channel of the hub, can be done more easy with an elastically deformable filter unit.

Alternatively or additionally, the surrounding material in which the sheet material is fixated may be of a thermoplastic material. Advantageously, the sleeve material can be a thermoplastic elastomer. Using a thermoplastic elastomer (TPE) provides the advantages of an elastic material. The hub material can be of a thermoplastic material. Moreover, a thermoplastic material is known to be used for medical applications; specific medical grades are available for each different type of thermoplastic elastomeric material.

Furthermore, thermoplastic elastomers allow for efficient and cost-effective manufacturing methods. The ease with which thermoplastic elastomers can be formed and shaped provides for easy, consistent and cost-effective mass production of the tubular sleeve.

In general there are six generic classes of commercial TPEs namely styrenic block copolymers, TPS (TPE-s); thermoplastic polyolefin elastomers, TPO (TPE-o); thermoplastic Vulcanizates, TPV (TPE-v or TPV); thermoplastic polyurethanes, TPU (TPU); thermoplastic copolyester, TPC (TPE-E) and thermoplastic polyamides, TPA (TPE-A). Preferably the tubular sleeve of the present invention is made out of thermoplastic styrenic elastomers (TPE-s) since they provide good adhesion to the woven filter material and hence excellent fixation of the filter within the tubular sleeve so that the filter can withstand the applied injection pressure of the drug.

Advantageously, the thermoplastic elastomer, in particular when used as sleeve material, may have a Shore A hardness of between 85 and 105, preferably between 90 and 100, more preferably between 94 and 97 according to standard ISO 868. Thermoplastic elastomers, especially thermoplastic styrenic elastomers, having a Shore A hardness within said ranges provide excellent sealing between the filter and the tubular sleeve as well as between the tubular sleeve and the hub. Shore Hardness scales measure the resistance of a material to indentation. The hardness of a material is tested using a durometer device. This measures the depth of an indentation in the material created by a given force on a standardised presser foot. A higher number indicates greater resistance to indentation so therefore a harder material. There are several scales of durometer used for materials with different properties, however in engineering plastics Shore A and Shore D are most commonly used. The Shore A scale is used for measuring the hardness of softer, more flexible materials. Shore A “0” denotes extremely soft, gel like materials such as silicones, while semi-rigid plastics will be measured at the highest end of the scale around 90-95A.

Further advantageously the thermoplastic elastomer may have a Melt Flow Rate of between 15 and 20 g/10 min, preferably between 16 and 18 g/10 min according to ISO 1133. A high flow rate avoids or diminished air being trapped around the filter during moulding which can cause unsatisfactory fixation between filter and sleeve resulting in potential leakage.

The filter sheet can be woven from any type of plastic material such as polyamide, PET, PA, PP. Advantageously, the sheet of woven filter material may be woven from a polyamide yarn. Polyamide yarn is easy to process, and allows for an efficient and cost-effective mass production of the woven filter. Furthermore, polyamide yarn is known to be able to comply with requirements for medical material.

Advantageously, the sheet of woven filter material may be a twill weave filter. A twill weave material wherein two yarns are used, warp and weft is found to have the best filter characteristics for filtering particles of a predefined size, in particular a twill weave material wherein the warp mesh count is between 300 and 350 threads/cm and the weft mesh count is between 250 and 300 threads/cm. More specifically, a twill weave material is found to have the best filter results according to USP789 (Particulate Matter in Ophthalmic Solutions test) being able to provide a filter efficiency of 97-98%. Alternatively, more yarns may be used for the twill weave filter as well.

The woven filter sheet may have a mesh size of between 2 and 150 μm depending on the size of the particulate contaminants to be filtered. In most applications a mesh size of below 20 μm or even below 5 μm is used. Especially when fine particle material needs to be filtered out, such as in ophthalmic solutions, a mesh size of about 5 μm may be required.

The surrounding material may be connected to the sheet of woven filter material by overmoulding to form a sheet integrated to the surrounding material. As such, an integrated filter unit of sheet of filter material and sleeve material can be obtained. Alternatively, a hub with an integrated sheet of filter material can be obtained. Injection overmoulding is a manufacturing technique suitable for an efficient and cost-effective mass production of the filter unit. Furthermore, a sheet of woven filter material is particularly suited to be overmoulded, as molten material can flow through meshes of the sheet of woven filter material, in particular through meshes of the outer edge of the sheet. After solidification, the sleeve material protrudes through the outer edge of the sheet of filter material, in particular through the meshes of the sheet of woven material. As such, a firm and reliable connection can be obtained between the filter material and the tubular sleeve, that may prevent leakage between the filter material and the sleeve. The final product, where part of the molten material has solidified whilst in between the meshes of the sheet of the woven filter material, comprises a sheet of woven filter material which is thoroughly fixated in the tubular sleeve. In particular, mould material can be flown through meshes of an outer edge of the sheet of filter material, such that the outer edge is integrally joined to the moulded surrounding material of the sleeve or hub. By using injection moulding, in a single manufacturing step, the filter unit can be made, providing for a cost effective manufacturing of the filter unit, or hub with an integrated filter unit. The filter unit comprising the sheet fixated to the sleeve, can then be inserted in the channel of the hub to provide a filter needle.

Instead of moulding or overmoulding, any other process may be used that allows melting of the tubular sleeve material such that it can flow through meshes of the filter material, such that, after solidification, the sleeve material protrudes through the filter material, in particular through meshes of the filter material. For example, local melting of the material e.g. by using a laser source, can be used.

The sheet of filter material can be positioned in the mould and the hub material can be moulded over it. The hub material then flows through meshes of the filter material. Then, the hub of the filter needle is directly moulded with the sheet of filter material directly fixated in the hub material, providing the hub in a one-step manufacturing mould process.

Advantageously, the sheet of filter material may be integrated to the surrounding material, by forming it by injection moulding via a double injection gate. Alternatively or additionally, each injection gate of the double injection gate may be positioned at a same distance from the sheet of woven filter material. Using a double injection gate results in less deformations being present in the filter unit. Also, by using a double injection gate, with one injection gate at each side of the filter material, a more even distribution of the moulded material can be obtained, as well as a more thorough flowing of the mould material through meshes of the filter at an outer edge of the filter, thereby providing for a more robust connection of the filter sheet to the sleeve or the hub of moulded material, as more of the surrounding material protrudes through the sheet of filter material. A double injection gate provides the advantage over a single injection point of even pressure on the filter sheet; this prevents folding of the outside of the filter which leads to a strong binding between filter and tubular sleeve and prevents leakage. As such, a double injection gate is preferred in order to enclose the entire circumference of the outer edge of the filter. Enclosing the entire circumference can be achieved by injecting an equal amount of mould material through each injection gate of the double injection gate.

Other manufacturing processes can however be used to connect the tubular sleeve to the sheet of woven material and ensure protrusion of the sheet material through the sheet of woven material. The tubular sleeve or the hub can for example be provided in two parts wherein the sheet of woven material is connected to one of the two parts by welding, such as point welding, or line welding, before connecting the two parts with one another. For example, the sheet of woven filter material can be welded to a first part of the hub or tubular sleeve. Then, a second part of the hub or tubular sleeve can be connected, e.g. by welding as well, to the first part of the hub or tubular sleeve respectively. As such, a firm engagement between the parts of the hub or tubular sleeve and the sheet of filter material can be obtained as well, though requiring more handling and manufacturing steps. Plastic material can be welded, e.g. by thermo-welding. Other connection methods can be used as well, such as adhesives compliant for medical use. Also, welding of the filter material to the hub or to the sleeve can be envisaged by positioning the sheet of filter material in the hub or sleeve, and welding the sleeve material until it locally flows such that it can flow through meshes of the sheet of filter material, such that after solidification the sheet of filter material is integrated to the hub or sleeve. In such a method, the hub or sleeve can be provided as a single part.

Advantageously, the filter unit comprising the sleeve may be symmetric with respect to a symmetry plane through the sheet of woven filter material.

Alternatively or additionally, the filter unit with the sleeve may be fitted into the channel of the hub by means of press-fitting. Press-fitting is an easy way to fixedly position the filter unit into the channel of the hub. Press-fitting does not require any additional materials such as glue or additional steps such as plastic welding and can be performed by anyone. Preferably, in neutral or rest position, an outer diameter of the tubular sleeve is somewhat larger than an inner diameter of the channel of the hub. Upon insertion of the filter unit into the channel, the filter unit can be slightly compressed, and when in position in the channel, can be released to its neutral position, due to it being elastically deformable. As such, tight press-fitting of the filter unit in the channel can be obtained, obviating any leakage between the sleeve wall and the channel wall.

Direction of the mounting of the tubular sleeve into the channel of the hub is not important since the filter efficiency is the same in both directions.

Alternatively or additionally, the height of the tubular sleeve may be smaller than the diameter of the tubular sleeve. As such a relatively small and compact filter unit is provided, which is advantageous in view of manufacturing costs, materials costs and simply usage of space. Also, such a shape with a smaller height than a diameter, misalignment of the filter unit into the channel, or mis-positioning of the filter unit into the channel may be obviated.

Alternatively or additionally, the filter unit may be mounted in the channel of the hub adjacent the distal end of the hub towards a needle opening. As such, the filter material is near the needle opening, an end of the needle opening into the channel of the hub, allowing an efficient barrier for particles.

Alternatively or additionally, between the sheet of woven filter material and the needle opening a chamber may be formed having a conically shaped wall. A conically shaped wall may positively influence the fluid flow characteristics of the fluid flowing through the hub of the needle. The conically shaped wall may for example prevent the formation of dead zones, where fluid is near stationary, or may reduce turbulence, or may reduce an accumulation of air bells present in the fluid.

Alternatively or additionally, a locking element may be provided in the channel of the hub. For example, the locking element may be a circumferential rim. A locking element, such as a circumferential rim, may provide additional assurance with relation to the fixation of the filter unit in the channel of the hub. The locking element may also be embodied as a shoulder or a rib, or as multiple radially protruding elements. Many variants are possible. In particular, when the needle is used for sucking up fluid into the syringe, the locking element may prevent displacement of the filter unit due to the force or velocity of the fluid flow. Further, when inserting the filter unit into the channel, such a locking element may provide a tactile feedback to the user, or robot used for assembling the filter unit into the channel, when the filter unit is pushed over the locking element to fit into the channel. The user, or robot arm, may then feel that the filter unit is in position.

According to an aspect of the invention, there is provided a syringe comprising a syringe body for containing a medical solution, comprising a filter needle according to any of the above.

According to an aspect of the invention there is provided an assembly of a needle hub having a proximal end arranged for removable connection to a syringe body, and having a distal end to which a needle is mounted, the hub provided with a channel between the proximal end and the distal end, for when the syringe body is connected, establishing a fluid connection between the syringe body and the needle, wherein in the channel a locking element is provided, and a filter unit, wherein the filter unit comprises a sheet of woven filter material fixated in a tubular sleeve, wherein the filter unit is positioned in the channel between the locking element and the needle.

According to an aspect of the invention, there is provided a filter unit for use in the filter needle, in the syringe, or in the assembly as described hereinabove, wherein the filter unit comprises a sheet of woven filter material fixated in a tubular sleeve, wherein an outer edge of the sheet of woven filter material is fixated in the tubular sleeve, wherein the sleeve material protrudes through the outer edge of the sheet of woven filter material. According to an aspect of the invention, there is provided for a hub with an integrated sheet of filter material for use in a filter needle.

According to an aspect of the invention there is provided a method for manufacturing a filter unit for use in the filter needle, or for manufacturing a hub with a filter unit, the method comprising providing a sheet of woven filter material, placing the sheet of filter material in a mould, overmoulding the sheet of woven filter material with thermoplastic material, as surrounding material, such that the thermoplastic material is injection moulded through an outer edge of the sheet of woven material and protrudes through the sheet of filter material after solidification, in particular through meshes of the sheet of filter material. Preferably, the entire outer edge of the sheet of woven material is enclosed by the tubular sleeve. The surrounding material can be hub material or sleeve material. As such, depending on the mould provided, the filter unit with the sleeve and integrated filter material or the hub with integrated filter material can be manufactured.

Advantageously, overmoulding may comprise using two injection gates, each injection gate at an opposite side of the filter sheet.

According to an aspect of the invention, there is provided a kit of a filter unit as described hereinabove, and a needle hub having a proximal end arranged for removable connection to a syringe body, and having a distal end to which a needle is mounted, the hub provided with a channel between the proximal end and the distal end. The kit may further comprise a syringe body.

According to an aspect of the invention, there is provided the use of the syringe as described hereinabove either to draw up a medical solution into the syringe or to inject a medical solution into a medical patient. More specifically the syringe as described hereinabove can be used for hypodermic solutions, more particularly for ophthalmic solutions.

Further advantageous embodiments are represented in the subclaims.

### BRIEF DESCRIPTION OF THE DRAWINGS

The disclosure is further elucidated by means of a schematic drawing. In the drawing the following figures are shown.

FIGS. 1A and 1B show a cross-sectional view of a filter needle according to an embodiment of the invention,

FIGS. 2A, 2B and 2C show different views of a filter unit according to an embodiment of the invention,

FIG. 3 shows a view of a filter unit FIGS. 4A, 4B and 4C show the insertion of a filter unit into a hub according to an embodiment of the invention,

FIGS. 5A, 5B and 5C show the insertion of a filter unit into a hub according to another embodiment of the invention,

FIG. 6A shows a cross-sectional view of a filter needle according to another embodiment of the invention, FIG. 6B shows a detail of FIG. 6A,

FIG. 7 shows a cross-sectional view of a filter needle according to another embodiment of the invention.

### DETAILED DESCRIPTION OF THE DRAWINGS

The figures are given by way of schematic representations of embodiments of the disclosure. Like features are denoted with the same or similar reference numbers. The figures are not necessarily drawn to scale and are to be seen as schematic.

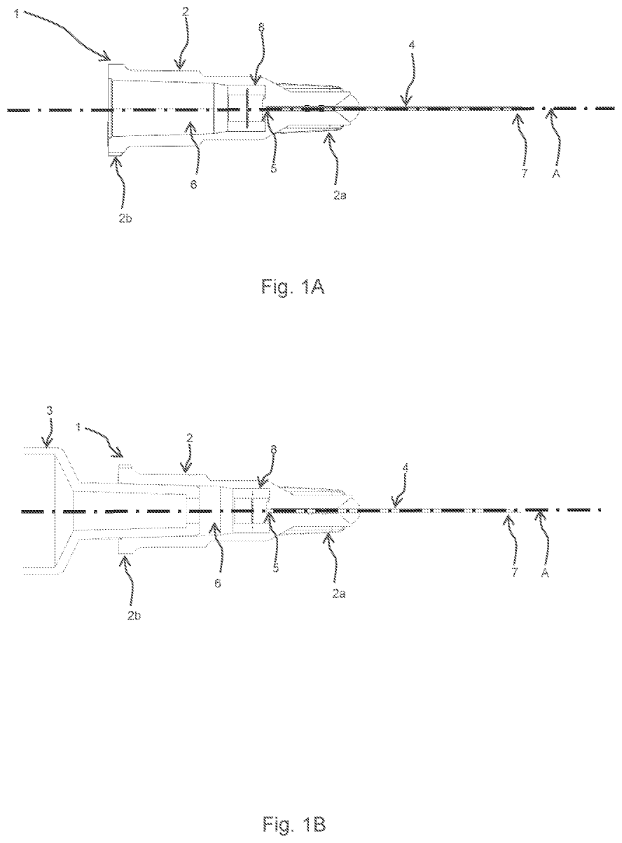

FIGS. 1A and 1B shows a cross-sectional view of a filter needle 1 according to an embodiment of the invention. The filter needle 1 comprises a hub 2 and a needle 4. The needle 4 is mounted in a distal end 2a of the hub 2. A syringe body 3, shown in FIG. 1B, can be connected to a proximal end 2b of the hub 2, e.g. by screwing or clamping or a bayonet connection. The hub 2 is provided with a channel 6 which establishes a fluid connection between the syringe body and the needle 4 when the syringe body is connected to the hub 2. The channel 6 connects the proximal end 2b of the hub 2 with the distal end 2a of the hub, and thus, in use, a fluid connection between the syringe body connected to the proximal end 2b and the needle 4 inserted in the distal end 2a is obtained. An end of the needle 4 ends at the channel 6 thus forming a needle opening 5 in the channel 6. As such, the channel 6 extends between the proximal end 2b and the needle opening 5 of the distal end 2a. An opposite end 7 of the needle 4 is arranged to be inserted into a patient and to allow fluid to exit from the needle 4. Alternatively, fluid can be sucked up via the end 7 of the needle 4 and then flows into the syringe body. However, the filter needle 1 is intended for one-way use only, i.e. either for injection fluid or either for sucking up fluid. After a single use of the filter needle 1, the filter needle 1 is disposed of.

The hub 2 extends along a central longitudinal axis A, and is preferably circular symmetric along the said axis. Advantageously, the needle 4 is arranged to the hub 2 such that a central longitudinal axis of the needle coincides with a central longitudinal axis of the hub 2, hereinafter all being referred to as the axis A. Also, the channel 6 is provided in the hub 2 having a central longitudinal axis coinciding with the longitudinal axis A of the hub 2.

A filter unit 8 is mounted in the channel 6 of the hub 2. The filter unit 8 is arranged to filter a solution, in particular a fluid solution, passing through the channel 6. More specifically, the filter unit 8 is arranged to prevent particulates or solid impurities from passing the filter unit 8. The filter unit 8 can hereto be used in two different manners. The filter unit 8 can, in a first manner, prevent particulates from travelling from the syringe body to the needle 4. As such, the filter unit 8 can prevent certain, possibly damaging or dangerous, particulates from being injected into a patient, when the needle 1 is used for injecting fluid. In a second manner, the filter unit 8 can prevent certain particulates from travelling from the needle 4 to the syringe body, when the needle 1 is used for sucking up fluid. As such, the filter unit 8 can prevent particulates from being sucked up from any kind of solution container into the syringe body. The filter unit 8 can for example prevent that any undesired precipitates formed in a medical solution end up in the syringe body or, later on, in a patient. It is stressed however that a specific filter unit 8 can only be used in one of the two manners described above. One same filter unit 8 can not be used to first filter a solution travelling from the needle 4 to the syringe body and to second filter the same or a different solution travelling from the syringe body to the needle 4. If one were to do so, the undesired particles filtered from the solution travelling towards the syringe body would be picked up by the same or a different solution travelling towards the needle 4. In FIGS. 1A and 1B, the filter unit 8 is mounted in the channel 6 of the hub 2 adjacent the distal end 2a towards a needle opening 5. It can however be appreciated that the hub 2 may be formed such that the filter unit 8 is mounted adjacent the proximal end 2b or the hub, or anywhere in between the proximal end 2b and the distal end 2a. The filter unit 8 extends along a central longitudinal axis B. Advantageously, after insertion of the filter unit 8 into the channel 6 the axis B coincides, or is at least aligned, with the axis A of the channel 6 and the hub 2.

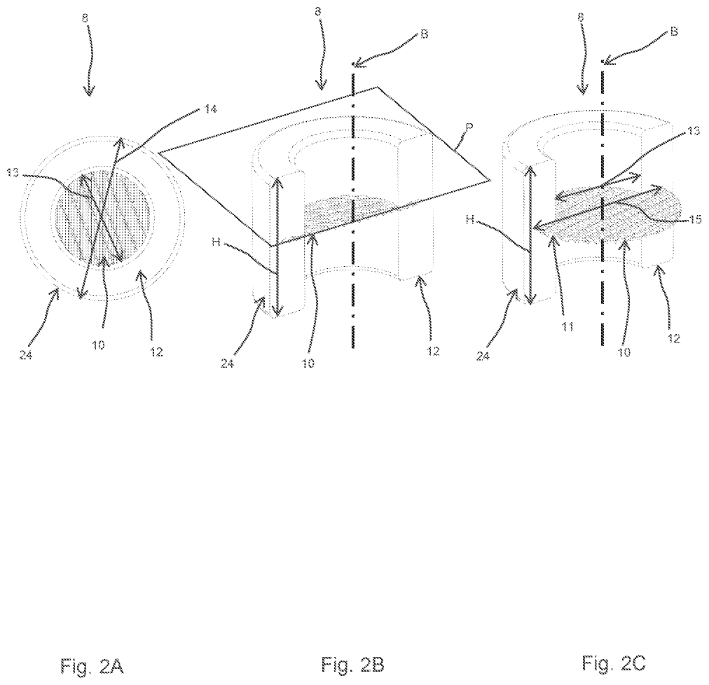

FIGS. 2A, 2B and 2C show different views of the filter unit 8 according to an embodiment of the invention. FIG. 2A shows a top view of the filter unit 8. The filter unit 8 comprises a sheet of woven material 10 fixated in a tubular sleeve 12. The tubular sleeve 12 has an inner diameter 13 and an outer diameter 14. The tubular sleeve 12 also has a height H, which can be seen on FIGS. 2B and 2C. In a similar fashion as the sheet of filter material is fixated to the sleeve material, by sleeve material protruding through meshes of the filter material, the sheet of filter material can be directly engaged to the hub material. Then, the outer diameter of the sheet of filter material is larger than an inner diameter of the channel 6 of the hub 2, to provide for sufficient filter material through which the hub material can flow.

The tubular sleeve 12 is made from an elastic material. This allows the tubular sleeve 12 to stretch or compress under stress, the tubular sleeve 12 being elastically deformable. A useful effect hereof arises when the filter unit 8 is to be mounted in the channel 6 of the hub 2 as seen in FIGS. 1A and 1B. Due to the tubular sleeve 12 being elastically deformable, it may fill up any irregularities or other impurities in an inner wall 61 of the channel 6 of the hub 2 and thereby sealingly close off the channel 6. The elastic deformable material of the tubular sleeve 12 may cause any small openings present between an outer wall 24 of the tubular sleeve 12 and the inner wall 61 of the channel 6 of the hub 2 and/or small tears present in the tubular sleeve 12 to close, thus preventing any fluid to leak past the filter. Another advantage of the elastically deformable tubular sleeve 12 is that the elastic tubular sleeve 12 can be press-fitted into the channel 6, which will be discussed more in detail in view of FIG. 4C.

The tubular sleeve 12 can be made from a thermoplastic elastomer, preferably a thermoplastic elastomer which has a Shore A hardness of between 85 and 105. Using a thermoplastic elastomer provides the advantages of an elastic material described herein. Furthermore, thermoplastic elastomers allow for efficient and cost-effective manufacturing methods. The ease with which thermoplastic elastomers can be formed and shaped provides for easy, consistent and cost-effective mass production of the tubular sleeve 12.

As mentioned above, the filter unit 8 also comprises a woven filter 10. The woven filter 10 filters particles of a predefined size out of a solution passing therethrough. Using a woven filter is known to bear some advantages in relation to using, for example, a membrane filter. A woven material is, for example, less sensitive to tearing and creates a lower pressure loss than a membrane filter, as such a woven filter may be more resistant against the pressure and/or velocity of the fluid flowing through the channel because it provides a higher fixation force with the material, in particular TPE of the tubular sleeve. The characteristics of a woven material depend on how the material is woven i.e. which type of weave is used. The woven filter 10 is manufactured according to a twill type weave. A twill weave material is found to have the best filter characteristics in view of the present application. Advantageously, a twill weave material using two yarns having a specific warp and weft mesh count can be used for filtering efficiency. Also, the size of the meshes of the woven material is determined in function of the particle size that is to be filtered out of the solution. More specifically, a twill weave material is found to have the best filter results according to USP789. Preferably, the woven filter 10 is woven from a polyamide yarn. Polyamide yarn is easy to process, and allows for an efficient and cost-effective mass production of the woven filter 10.

Referring now to FIGS. 2B and 2C, it can be seen that the woven material 10 is positioned halfway of the height H of the tubular sleeve 12. Otherwise, the filter unit 8 is symmetric with respect to a symmetry plane P through the sheet of woven material 10. The advantage hereof is that it doesn't matter in which direction the filter unit 8 is inserted into the hub 2. Someone attempting to insert the filter unit 8 into the hub 2 therefore doesn't have to check whether the filter unit 8 is positioned correctly, resulting in an easy and effortless insertion of the filter unit 8 into the hub 2, as long as the tubular wall 12 is aligned with the channel, such that a longitudinal axis B of the filter unit may coincide with a longitudinal axis A of the channel 6.

Referring now to FIGS. 2B and 2C, it can be seen that the woven filter 10 is advantageously disc shaped. Furthermore, it can be seen that an outer diameter 15 of the woven filter 10 is larger than the inner diameter 13 of the tubular sleeve 12. An outer edge 11 of the woven filter 10 is fixated in the tubular sleeve 12. The result hereof is that leakages due to imperfect sealing between the edge of a filter material and a tubular sleeve can be prevented. In order to fixate the woven filter 10 in the tubular sleeve 12, injection moulding can be used. More specifically, the tubular sleeve 12 can be overmoulded onto the woven filter 10. In an advantageous manner, the plastic material used to injection mould the tubular sleeve 12 will, when molten, flow into the meshes of the outer edge 11 of the woven filter 10. Thus, upon solidification of the molten material, the woven filter 10 will be securely fixated in the tubular sleeve 12.

As can be seen in FIG. 2c, preferably an annular outer portion 11 of the filter 10 is fixedly engaged to the tubular sleeve 12.

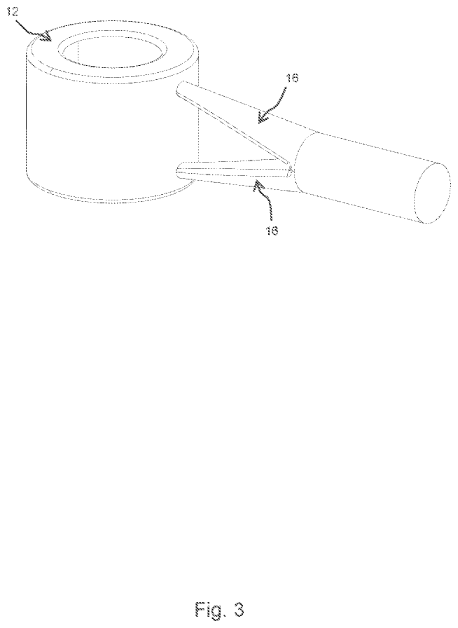

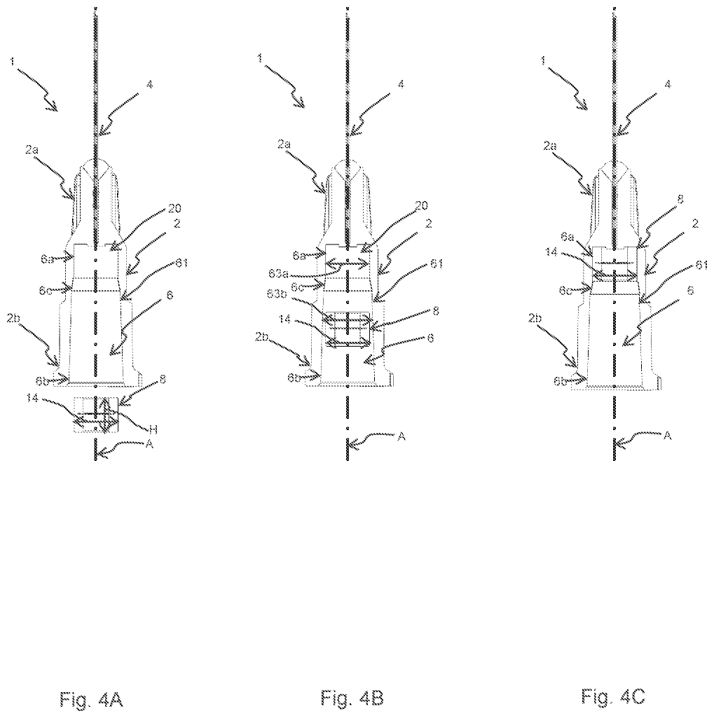

FIG. 3 shows an example of how a tubular sleeve 12 can be injection moulded. Two injection gates 16 are shown, wherein each injection gate 16 is positioned at about an equal distance from the woven filter 10 (not shown here). Before injecting the molten material into the mould, the woven filter 10 is positioned in the mould. This can for example be accomplished by clamping the woven filter 10 between two cores. Hereafter, the molten material is injected into the mould through the two injection gates 16. The advantage of using two injection gates 16 positioned at an equal distance from the woven filter 10 is that the two flows of molten material coming from the injection gates 16 will meet each other substantially at the woven filter 10. In other words, the molten material will flow against and in between the yarns of the woven filter 10 substantially equally from each side of the woven filter 10. The net result of these two flows reaching the woven filter 10 at a substantially equal point of time is that the pressure applied by the molten material on each side of the woven filter 10 is substantially equal. As such, the woven filter 10 is barely displaced by the flow of molten material during the injection moulding process. This prevents a bad connection, and subsequently leakages, between an edge of the woven filter 10 and the tubular sleeve 12 which can otherwise occur due to the edge of the woven filter 10 being pushed aside by the flow of molten material. In conclusion, providing two injection gates 16 positioned at an equal distance from the woven filter 10 results in a better fixation between the woven filter 10 and the tubular sleeve 12, and thus prevents leakages due to a bad fixation between the woven filter 10 and the tubular sleeve 12.

The injection moulding process allows for an efficient and cost effective mass production of the tubular sleeve 12. As mentioned above, the woven filter 10 has the same advantage due to being woven from a polyamide yarn. These steps mainly form the production process for the filter unit 8 as a whole. As such, the filter unit 8 is in itself efficient and cost effective to mass produce.

FIG. 3 shows two injection gates 16 positioned each at an opposite side of the sheet of filter material, for example at an equal distance from the woven filter 10. It may however be appreciated that the tubular sleeve 12 can be injection moulded using any number of injection gates 16 positioned anywhere with relation to the woven filter 10 whilst remaining within the scope of the appended claims.

Alternatively to the injection moulding process, the filter unit may be assembled e.g. by welding such as thermo-welding or by medical use compliant adhesives. For example, the sleeve 12 may be provided in two parts, a first part for being arranged at one side of the filter, and a second part for being arranged at the opposite side of the filter. The filter, in particular an outer edge thereof, can then be glued or welded, or point-welded or otherwise connected to first part, such that the outer edge of the filter overlaps the tubular sleeve. Then, the second part can be connected to the filter and the first part, e.g. by welding or gluing as well. Thus, a firm connection between the first part, the filter and the second part can be obtained to form the filter unit. It is recognised though that this method involves more steps, and, thus, more risks on leakage.

Now turning to FIGS. 4A, 4B and 4C, a schematic example is shown of how the filter unit 8 can be inserted into the hub 2. FIG. 4A shows that the filter unit 8 is inserted into the channel 6 of the hub 2 via the proximal end 2b of the hub 2. FIG. 4B shows that the an inner diameter 63b of the channel 6 is larger than the outer diameter 14 of the tubular sleeve 12 of the filter unit 8 near the proximal end 2b of the hub 2. As such, the filter unit 8 can move freely in the channel 6 near the proximal end 2b of the hub. As seen in FIG. 4B, the size of the filter unit 8 can be sufficiently large to prevent rotation of the filter unit 8 around an axis not equal to a longitudinal axis A of the hub 2 and the needle 4 and/or to prevent misalignment of the filter unit 8 in the channel 6. In this example, by providing a height H of the filter unit 8 that is smaller than the outer diameter 14 of the filter unit 8, it can be obviated that the filter unit 8 is accidently rotated during insertion of the filter unit 8 into the hub. This accidental rotation of a filter unit could otherwise be quite harmful. An accidently rotated filter unit may effectively block fluid from flowing through the channel 6 or, even worse, an accidently rotated filter might provide openings for the fluid to flow through the channel 6 without getting filtered. The prevention of accidental rotation of the filter unit 8 has another advantage, namely that a user inserting the filter unit 8 into the hub 2 does not have to be particularly careful as to correctly move the filter unit 8 through the channel 6. The user may in fact just simply push the filter unit 8 through the channel 6. Instead of a user inserting the filter unit 8 into the channel 6, an automated process may be used as well, e.g. using a robot pushing the filter unit 8 into the channel 6. In the latter situation, the shape of the filter unit 8 can be advantageous to prevent misalignment.

The diameter of the channel 6, in this example, decreases towards the distal end 2a of the hub 2. Near the proximal end 2a of the hub 2, an inner diameter 63a of the channel 6 is slightly smaller than the outer diameter 14 of the tubular sleeve 12 of the filter unit 8. The channel 6 may thus have a proximal part 6b with a larger diameter 63b, a distal part 6a with a smaller diameter 63a and a tapered part 6c connecting the proximal part 6b and the distal part 6a. The distal part 6a here forms a chamber 20 in which the filter unit 8 can be received. As such, and due to the elastic properties of the tubular sleeve 12, the filter unit 8 can be press-fitted in the channel 6 when in the position illustrated by FIG. 4C. An advantage of the filter unit 8 being press-fitted in the channel 6 is that any openings, due to irregularities both on the outer wall 24 of the tubular sleeve 12 or on the inner wall 61 of the channel 6, are pressed shut, thus preventing fluid to leak through the interface between the channel 6 and the tubular sleeve 12. This effect may be strengthened by any pressure applied by the fluid on the tubular sleeve 12.

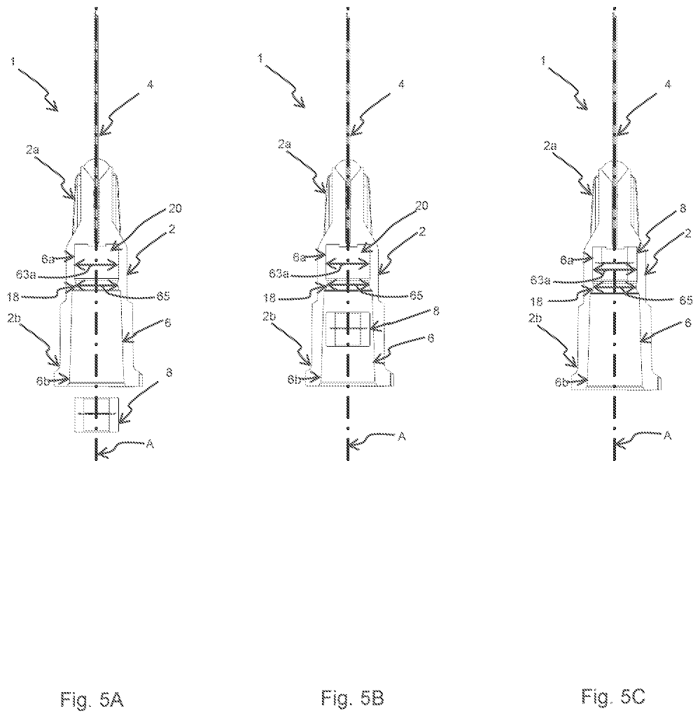

FIGS. 5A, 5B and 5C show the insertion of the filter unit 8 into another embodiment of the hub 2. The hub 2, in this example, comprises a locking element 18, illustrated here as a circumferential rim 18, connecting the proximal part 6b and the distal part 6a. The circumferential rim 18 provides an inner diameter 65 of the channel 6 which is smaller than the inner diameter 63a of the channel 6 near the proximal end 2a of the hub. In other words, the circumferential rim 18 provides the smallest inner diameter along the channel 6. An advantage of the circumferential rim 18 is that the circumferential rim 18 further limits translational movement of the filter unit 8 in a direction along the longitudinal axis A, when the filter unit 8 is positioned near the proximal end 2a of the channel 6 as shown in FIG. 5C. In view of this advantage, the circumferential rim 18 strengthens the fixating effect of the filter unit 8 being press-fitted into the channel 6. It may be appreciated, however, that the additional fixation of the filter unit 8 provided by the circumferential rim 18 is purely optional, and that the press-fitting of the filter unit 8 on its own may be sufficient in order to keep the filter unit 8 in place. Another advantage of the circumferential rim 18 is that a user, or an automated robot, inserting the filter unit 8 in the channel 6, will have to press slightly harder to get the filter unit 8 to move past the circumferential rim 18, and will feel a slight pop, as a tactile feedback, when the filter unit 8 has passed the circumferential rim 18. This tactile feedback informs the user that the filter unit 8 is pushed all the way through to where the filter unit 8 should be. As such the user can rest assured that the filter unit 8 is pushed far enough into the channel 6.

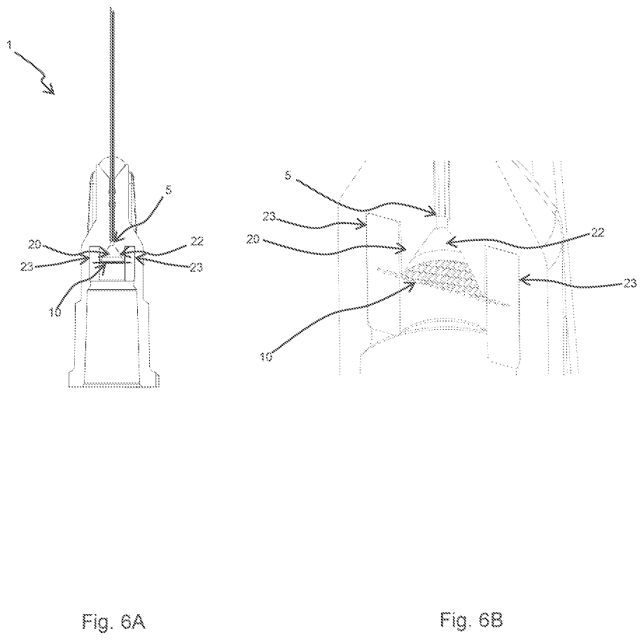

FIGS. 6A and 6B show a filter needle 1 according to another embodiment of the invention. The hub 2, in this example, comprises a chamber 20 having a conically shaped wall 22. Further, the chamber 20 also provides for a receiving slit 23 to receive the tubular wall 12 of the filter unit 8 therein. As such, the conically shaped wall 22 can extend towards the filter material 10, when the filter unit 8 is inserted into the chamber 20. The conically shaped wall 22 extends from the needle opening 5 towards the woven filter 10. Due to the conically shaped wall 22, a solution travelling between the needle opening 5 and the woven filter 10 does not encounter abrupt changes of the cross sectional area through which the solution flows. Abrupt changes of this cross sectional area disrupts fluid flow, causing dead zones where solution is near stationary. These dead zones could for example occur near the needle opening 5 as shown in FIGS. 1A and 1B, where the cross sectional area abruptly changes between the filter unit 8 and the needle opening 5. Abrupt changes of this cross sectional area can also stimulate turbulent fluid flow, which is less efficient and less predictable than a laminar fluid flow.

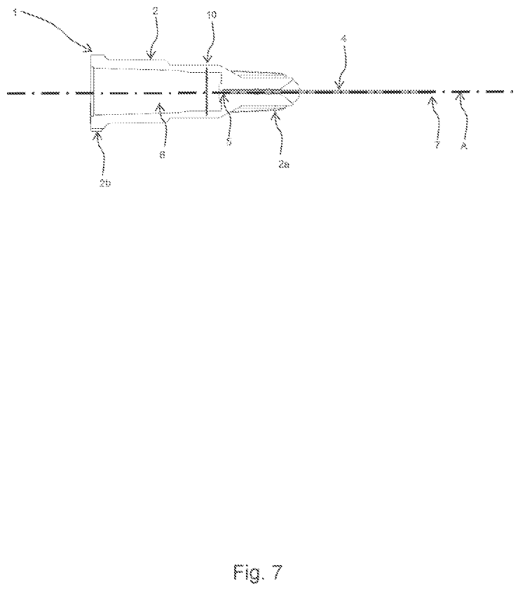

FIG. 7 shows a cross-sectional view of a filter needle 1 according to another embodiment of the invention. The hub 2, in this example, comprises a sheet of filter material 10, positioned in the channel 6 of the hub 2. The sheet of filter material 10 is here fixated to the hub 2 by placing the sheet of woven filter material 10 in a hub mould, and overmoulding the sheet of woven filter material 10 with hub material, such that the hub material is injection moulded through an outer edge 11 of the sheet of woven filter material 10, in particular through meshes of the sheet of woven filter material 10. Alternatively, the sheet of filter material can be welded directly to the hub. The hub material preferably is a thermoplastic material. The sheet of woven filter material 10 is arranged to filter a solution, in particular a fluid solution, passing through the channel 6. More specifically, the sheet of woven filter material 10 is arranged to prevent particulates or solid impurities from passing the sheet of woven filter material 10 fixated in the hub 2. The sheet of woven filter material 10 fixated in the hub 2 can hereto be used in two different manners. The sheet of woven filter material fixated in the hub 2 can, in a first manner, prevent particulates from travelling from the syringe body to the needle 4. As such, the sheet of woven filter material 10 fixated in the hub 2 can prevent certain, possibly damaging or dangerous, particulates from being injected into a patient, when the needle 1 is used for injecting fluid. In a second manner, the sheet of woven filter material 10 fixated in the hub 2 can prevent certain particulates from travelling from the needle 4 to the syringe body, when the needle 1 is used for sucking up fluid. As such, the sheet of woven filter material 10 fixated in the hub 2 can prevent particulates from being sucked up from any kind of solution container into the syringe body. The sheet of woven filter material 10 fixated in the hub 2 can for example prevent that any undesired precipitates formed in a medical solution end up in the syringe body or, later on, in a patient. In FIG. 7, the sheet of woven filter material 10 is fixated in the hub 2 near the distal end 2a towards a needle opening 5. It can however be appreciated that the hub 2 may be formed such that the sheet of woven filter material 10 is fixated adjacent the proximal end 2b of the hub, or anywhere in between the proximal end 2b and the distal end 2a. The filter needle is primarily a so-called hypodermic needle. The filter unit can be used with needles of varying length and thickness, so called Gauge, ranging mostly from 18 to 34 Gauge.

The syringe of the present invention and its filter needle can be used either to suck up liquid into the syringe or to inject liquid from the syringe. Although the main application of the syringe and its filter needle are in the medical field it can be used for non-medical applications as well. The most important application of the syringe and its filter needle is to draw up medical solution from a vial into the syringe or to inject a medical solution into a patient with the purpose of effectively filtering out particulate contaminants present in the medical solution. The filter needle is particularly suitable to filter out from medical solutions particulate contaminants primarily having a particle size of larger than 5 μm provided the mesh size of the woven filter material is 5 μm. Therefore the syringe and its filter needle are particularly suitable for ophthalmic applications but also other medical applications wherein it is important to avoid the risk of having particles injected into the human body can be envisaged.

The term “medical solutions or medical liquid or medical fluid” as used throughout herein is intended to refer to any solution intravenously or intramuscularly fed to a patient, including medication injected by a hypodermic syringe.

Herein, the invention is described with reference to specific examples of embodiments of the invention. It will, however, be evident that various modifications, variations, alternatives and changes may be made therein, without departing from the essence of the invention. For the purpose of clarity and a concise description features are described herein as part of the same or separate embodiments, however, alternative embodiments having combinations of all or some of the features described in these separate embodiments are also envisaged and understood to fall within the framework of the invention as outlined by the claims. The specifications, figures and examples are, accordingly, to be regarded in an illustrative sense rather than in a restrictive sense. The invention is intended to embrace all alternatives, modifications and variations which fall within the spirit and scope of the appended claims. Further, many of the elements that are described are functional entities that may be implemented as discrete or distributed components or in conjunction with other components, in any suitable combination and location.

Many variants are possible and are comprised within the scope of the following claims.

1. A filter needle comprising:
a hub having a proximal end arranged for removable connection to a syringe body, and having a distal end in which a needle is mounted, the hub provided with a channel between the proximal end and the distal end, for when the syringe body is connected, establishing a fluid connection between the syringe body and the needle, wherein a filter unit is mounted in the channel of the hub, the filter unit further including a sheet of woven filter material, wherein the sheet of woven filter material is fixated to a surrounding material, wherein the surrounding material protrudes through the sheet of woven filter material.

2. The filter needle according to claim 1, wherein the surrounding material is a hub material.

3. The filter needle according to claim 1, wherein the filter unit further includes a sleeve, preferably a tubular sleeve, wherein the surrounding material is a material of the sleeve.

4. The filter needle according to claim 1, wherein an outer edge of the sheet of woven filter material is fixated to the surrounding material, wherein the surrounding material protrudes through the outer edge of the sheet of woven filter material, preferably through meshes of the outer edge of the sheet of filter material.

5. The filter needle according to claim 3, wherein the sheet of woven filter material is positioned halfway of the height of the tubular sleeve.

6. The filter needle according to claim 1, wherein the sheet of woven filter material is disc-shaped.

7. The filter needle according to claim 3, wherein the filter unit, in particular the tubular sleeve, is elastic.

8. The filter needle according to claim 1, wherein the surrounding material, is of a thermoplastic material, preferably wherein the material of the tubular sleeve of the filter unit is of a thermoplastic elastomer, in particular of a thermoplastic styrenic elastomer.

9. The filter needle according to claim 8, wherein the thermoplastic elastomer has a Shore A hardness of between 85 and 105, measured according to standard ISO 868.

10. The filter needle according to claim 8, wherein the thermoplastic elastomer has a Melt Flow Rate between 15 and 20 g/10 min measured according to standard ISO 1133 at 230° C. and 2.16 kg.

11. The filter needle according to claim 1, wherein the sheet of woven filter material is woven from a polyamide or polyester or polypropylene yarn.

12. The filter needle according to claim 1, wherein the sheet of woven filter material is a twill weave filter.

13. The filter needle according to claim 1, wherein the surrounding material is integrated to the sheet of woven filter material by locally melting of the surrounding material.

14. The filter needle according to claim 1, wherein the sleeve is connected to the sheet of woven filter material by overmoulding to form an integrated filter unit or wherein the hub is connected to the sheet of woven filter material by overmoulding to form an integrated hub with sheet.

15. The filter needle according to claim 1, wherein the filter unit comprising the sleeve is formed by injection moulding via a double injection gate, or wherein the hub comprising the sheet of filter material is formed by injection moulding via a double injection gate.

16. The filter needle according to claim 15, wherein each injection gate of the double injection gate is positioned at another side from the sheet of woven filter material.

17. The filter needle according to claim 1, wherein the filter unit is symmetric with respect to a symmetry plane through the sheet of woven filter material.

18. The filter needle according to claim 3, wherein the outer diameter of the filter unit, in particular the outer diameter of the tubular sleeve, is larger than an inner diameter of the channel of the hub.

19. The filter needle according to claim 3, wherein the filter unit is fitted into the channel of the hub by means of press-fitting.

20. The filter needle according to claim 3, wherein the height of the tubular sleeve is smaller than the outer diameter of the tubular sleeve.

21. The filter needle according to claim 3, wherein the filter unit is mounted in the channel of the hub adjacent the distal end of the hub towards a needle opening.

22. The filter needle according to claim 1, wherein in the channel, adjacent a needle opening, a chamber is formed having a conically shaped wall.

23. The filter needle according to claim 3, wherein in the channel of the hub a locking element is provided for locking the filter unit, in particular the sleeve, in the channel.

24. The filter needle according to claim 23, wherein the locking element is a circumferential rim.

25. The filter needle according to, wherein a mesh size of the woven filter material is between 2 μm and 150 μm.

26. A syringe comprising a syringe body for containing a medical solution and a filter needle according to claim 1.

27. Assembly of a needle hub having a proximal end arranged for removable connection to a syringe body, and having a distal end to which a needle is mounted, the hub provided with a channel between the proximal end and the distal end, for when the syringe body is connected, establishing a fluid connection between the syringe body and the needle, wherein in the channel a filter unit is provided, wherein the filter unit comprises a tubular sleeve and a sheet of woven filter material fixated in a tubular sleeve wherein material of the sleeve protrudes through an outer edge of the sheet of wove filter material, wherein the filter unit is positioned in the channel.

28. The filter unit for use in the filter needle of claim 3, wherein the filter unit includes a sheet of woven filter material fixated in a tubular sleeve.

29. Method for manufacturing a filter unit for use in the filter needle of claim 3, the method comprising: providing a sheet of woven filter material; placing the sheet of woven filter material in a sleeve mould; overmoulding the sheet of woven filter material with thermoplastic material as sleeve material, such that the thermoplastic material is injection moulded through an outer edge of the sheet of woven filter material, in particular through meshes of the sheet of woven filter material.

30. Method according to claim 29, wherein overmoulding comprises using two injection gates.

31. Method for manufacturing a hub for use in the filter needle of claim 1, the method comprising: providing a sheet of woven filter material; placing the sheet of woven filter material in a hub mould; overmoulding the sheet of woven filter material with thermoplastic material as hub material, such that the thermoplastic material is injection moulded through an outer edge of the sheet of woven filter material, in particular through meshes of the sheet of wove filter material.

32. A kit of a filter unit according to claim 28, and of a needle hub having a proximal end arranged for removable connection to a syringe body, and having a distal end to which a needle is mounted, the hub provided with a channel between the proximal end and the distal end.

33. A kit according to claim 32, further comprising a syringe body.
